(19) United States
(12) Patent Application Publication
(10) Pub. No.: US 2016/0145244 A1
(43) Pub. Date: May 26, 2016

(54) CERTAIN PROTEIN KINASE INHIBITORS

(71) Applicants: Weibo Wang, Moraga, CA (US); Meiyu Geng, Shanghai (CN); Jian Ding, Shanghai (CN); Xingdong Zhao, Moraga, CA (US); Jing Ai, Shanghai (CN); Huajie Zhang, Chongqing (CN); Xia Peng, Shanghai (CN)

(72) Inventors: Weibo Wang, Moraga, CA (US); Meiyu Geng, Shanghai (CN); Jian Ding, Shanghai (CN); Xingdong Zhao, Moraga, CA (US); Jing Ai, Shanghai (CN); Huajie Zhang, Chongqing (CN); Xia Peng, Shanghai (CN)

(73) Assignee: FOCHON PHARMA, INC., San Leandro, CA (US)

(21) Appl. No.: 14/945,426

(22) Filed: Nov. 19, 2015

Related U.S. Application Data
(60) Provisional application No. 62/084,542, filed on Nov. 25, 2014.

(51) Int. Cl. C07D 405/14

(52) U.S. Cl. CPC C07D 405/14

(57) ABSTRACT

Provided are certain ALK inhibitors, pharmaceutical compositions thereof, and methods of use therefor.

This application claims the priority to the U.S. provisional application No. 62/084,542, filed on Nov. 25, 2014, which is incorporated herein by reference in its entirety.

### TECHNICAL FIELD

Provided are certain compounds or pharmaceutically acceptable salts thereof which can inhibit kinase activity of ALK and may be useful for the treatment of hyper-proliferative diseases like inflammatory, autoimmune disorders and cancer.

### BACKGROUND

Anaplastic lymphoma kinase (ALK), a member of the insulin receptor superfamily of receptor tyrosine kinases, has been implicated in oncogenesis in hematopoietic and non-hematopoietic tumors. The aberrant expression of full-length ALK receptor proteins has been reported in neuroblastomas and glioblastomas; and ALK fusion proteins have occurred in anaplastic large cell lymphoma. The study of ALK fusion proteins has also raised the possibility of new therapeutic treatments for patients with ALK-positive malignancies. (Pulford et al., Cell. Mol. Life. Sci. 61:2939-2953 (2004)).

Although ALK inhibitors were disclosed in the arts, e.g. WO 2004076412, WO 2008073687, there is a continuing need for compounds which may be useful for treating and preventing a disease which responds to inhibition of ALK and have at least one advantageous property selected from potency, stability, selectivity, toxicity, pharmacodynamics properties and pharmacokinetics properties as an alternative. In this regard, a novel class of ALK inhibitors is provided herein.

### DISCLOSURE OF THE INVENTION

Disclosed herein are certain novel triazine derivatives and pharmaceutical compositions thereof, and their use as pharmaceuticals.

In one aspect, disclosed herein is a compound of formula (I):

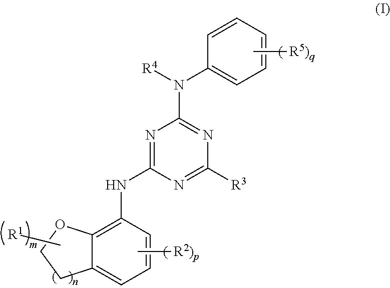

or a pharmaceutically acceptable salt thereof, wherein:

each R¹ is independently selected from hydrogen, halogen, hydroxyl, C₁₋₁₀ alkyl (such as C₁₋₆ alkyl), C₃₋₁₀ cycloalkyl (such as C₃₋₆ cycloalkyl), C₃₋₁₀ cycloalkyl-C₁₋₄ alkyl, heterocyclyl, heterocyclyl-C₁₋₄ alkyl, aryl, aryl-C₁₋₄ alkyl, heteroaryl, and heteroaryl-C₁₋₄ alkyl, wherein alkyl, cycloalkyl, and heterocyclyl are each unsubstituted or substituted with at least one substituent, such as one, two, three, or four substituents, independently selected from R^{6a}, and wherein aryl and heteroaryl are each unsubstituted or substituted with at least one substituent, such as one, two, three, or four substituents, independently selected from R^{6b};

each R² is independently selected from hydrogen, halogen, hydroxyl, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, heterocyclylcarbonyl, aryl, heteroaryl, aryl-C₁₋₄ alkyl, and heteroaryl-C₁₋₄ alkyl, wherein alkyl, alkenyl, alkynyl, cycloalkyl, and heterocyclyl are each unsubstituted or substituted with at least one substituent, such as one, two, three, or four substituents, independently selected from R^{6a}, and each aryl and heteroaryl is unsubstituted or substituted with at least one substituent, such as one, two, three, or four substituents, independently selected from R^{6b};

each R³ is independently selected from hydrogen, halogen, —CN, —NR⁷R⁸, and C₁₋₁₀ alkyl; wherein alkyl is unsubstituted or substituted with at least one substituent, such as one, two, three, or four substituents, independently selected from R^{6a};

each R⁴ is independently selected from hydrogen, halogen, —CN, C₁₋₁₀ alkyl, C₂₋₁₀ alkenyl, C₂₋₁₀ alkynyl, and C₃₋₁₀ cycloalkyl; wherein alkyl, alkenyl, alkynyl, and cycloalkyl are each unsubstituted or substituted with at least one substituent, such as one, two, three, or four substituents, independently selected from R^{6a};

or R³ and R⁴ together with the carbon atoms to which they are attached form a 5-6 membered ring containing 0, 1, 2 or 3 heteroatoms independently selected from oxygen, sulfur and nitrogen, and optionally substituted with 1-2 R^{6b} groups;

each R⁵ is independently selected from hydrogen, C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃; wherein alkyl, alkenyl, alkynyl, and cycloalkyl are each unsubstituted or substituted with at least one substituent, such as one, two, three, or four substituents, independently selected from R^{6a};

each R^{6a} is independently selected from C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —(CR⁹R¹⁰)ₜOR⁸, —(CR⁹R¹⁰)ₜNR⁷R⁸, —(CR⁹R¹⁰)ₜSR⁸, —(CR⁹R¹⁰)ₜS(O)ᵣR⁸, —(CR⁹R¹⁰)ₜCO₂R⁸, —(CR⁹R¹⁰)ₜCONR⁷R⁸, —(CR⁹R¹⁰)ₜNR⁷CO₂R⁸, —(CR⁹R¹⁰)ₜOCONR⁷R⁸, —(CR⁹R¹⁰)ₜNR⁷CONR⁷R⁸, —(CR⁹R¹⁰)ₜNR⁷SO₂NR⁷R⁸, —O(CR⁹R¹⁰)ₜNR⁷R⁸, —C(O)R⁷, —C(O)(CR⁹R¹⁰)ₜOR⁸, —C(O)(CR⁹R¹⁰)ₜNR⁷R⁸, —C(O)(CR⁹R¹⁰)ₜSR⁸, —C(O)(CR⁹R¹⁰)ₜS(O)ᵣR⁸, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃;

each R^{6b} is independently selected from R^{6a}, aryl, aryl-C₁₋₄ alkyl, heteroaryl, and heteroaryl-C₁₋₄ alkyl;

each R⁷ and each R⁸ are independently selected from hydrogen, C₁₋₁₀ alkyl, C₂₋₁₀ alkenyl, C₂₋₁₀ alkynyl, cycloalkyl, cycloalkyl-C₁₋₄ alkyl; heterocyclyl, heterocyclyl-C₁₋₄ alkyl, aryl, heteroaryl, aryl-C₁₋₄ alkyl, and heteroaryl-C₁₋₄ alkyl; wherein alkyl, alkenyl, alkynyl, cycloalkyl, and heterocyclyl are each unsubstituted or substituted with at least one substituent, such as one, two, three, or four substituents, independently selected from R^{6a}, and aryl and heteroaryl are each unsubstituted or substituted with at least one substituent, such as one, two, three, or four substituents, independently selected from R^{6b}; or

R⁷ and R⁸ together with the atom(s) to which they are attached form a heterocyclic ring of 4 to 7 members containing 0, 1, or 2 additional heteroatoms independently selected from oxygen, sulfur and NR¹¹, each R⁷ and R⁸ may be unsubstituted or substituted on a carbon or nitrogen atom with at least one substituent, such as one, two, or three substituents, selected from R¹²;

each R⁹ and each R¹⁰ are independently selected from: hydrogen, C₁₋₁₀ alkyl, C₂₋₁₀ alkenyl, C₂₋₁₀ alkynyl, cycloalkyl, cycloalkyl-C₁₋₄ alkyl, heterocyclyl, heterocyclyl-C₁₋₄ alkyl, aryl, heteroaryl, aryl-C₁₋₄ alkyl, and heteroaryl-C₁₋₄ alkyl; or

R⁹ and R¹⁰ together with the carbon atom(s) to which they are attached form a ring of 3 to 7 members containing 0, 1, or 2 heteroatoms independently selected from oxygen, sulfur and nitrogen, and optionally substituted with 1-2 R^{6a} groups;

each R¹¹ is independently selected from hydrogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —S(O)ᵣR⁷, —C(O)R⁷, —CO₂R⁷, —CO₂(CR⁹R¹⁰)ₜCONR⁷R⁸, and —C(O)NR⁷R⁸;

each R¹² is independently selected from halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —OR⁷, —NR⁷S(O)ᵣR⁸, —S(O)ᵣR⁷, —SR⁷, —S(O)₂OR⁷, —OS(O)₂R⁷, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CHF₂, —CF₃, —OCHF₂, and —OCF₃;

each m is independently selected from 0, 1 and 2;

each n is independently selected from 1, 2, and 3;

each p is independently selected from 0, 1, 2, and 3;

each q is independently selected from 0, 1, 2, and 3;

each r is independently selected from 1 and 2;

each t is independently selected from 1, 2, and 3.

In yet another aspect, the present disclosure provides pharmaceutical compositions comprising a compound of formula (I) and/or a pharmaceutically acceptable salt thereof and a pharmaceutically acceptable excipient.

In yet another aspect, the disclosure provides methods for modulating ALK, comprising administering to a system or a subject in need thereof, a therapeutically effective amount of a compound of formula (I) and/or a pharmaceutically acceptable salt thereof or pharmaceutical compositions thereof, thereby modulating said ALK.

The disclosure also provides methods to treat, ameliorate or prevent a condition which responds to inhibition of ALK comprising administering to a system or subject in need of such treatment an effective amount of a compound of formula (I) and/or a pharmaceutically acceptable salt thereof or pharmaceutical compositions thereof, and optionally in combination with a second therapeutic agent, thereby treating said condition.

Alternatively, the present disclosure provides the use of a compound of formula (I) and/or a pharmaceutically acceptable salt thereof in the manufacture of a medicament for treating a condition mediated by ALK. In particular embodiments, the compounds of the disclosure may be used alone or in combination with a second therapeutic agent to treat a condition mediated by ALK.

Alternatively, disclosed is a compound of formula (I) for treating a condition mediated by ALK.

Specifically, the condition herein includes but not limited to, is an autoimmune disease, a transplantation disease, an infectious disease or a cell proliferative disorder.

Furthermore, the disclosure provides methods for treating a cell proliferative disorder, comprising administering to a system or subject in need of such treatment an effective amount of a compound of formula (I) and/or a pharmaceutically acceptable salt thereof or pharmaceutical compositions thereof, and optionally in combination with a second therapeutic agent, thereby treating said condition.

Alternatively, the present disclosure provides the use of a compound of formula (I) and/or a pharmaceutically acceptable salt thereof in the manufacture of a medicament for treating a cell-proliferative disorder. In particular examples, the compounds of the disclosure may be used alone or in combination with a chemotherapeutic agent to treat a cell proliferative disorder.

Specifically, the cell proliferative disorder disclosed herein includes but not limited to, lymphoma, osteosarcoma, melanoma, or a tumor of breast, renal, prostate, colorectal, thyroid, ovarian, pancreatic, neuronal, lung, uterine or gastrointestinal tumor.

In the above methods for using the compounds of the disclosure, a compound of formula (I) and/or a pharmaceutically acceptable salt thereof may be administered to a system comprising cells or tissues, or to a subject including a mammalian subject such as a human or animal subject.

### CERTAIN TERMINOLOGY

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, CH₂O is equivalent to OCH₂.

The term “alkyl” refers to both branched and straight-chain saturated aliphatic hydrocarbon groups having the specified number of carbon atoms. Unless otherwise specified, “alkyl” refers to C₁-C₆ alkyl. For example, C₁-C₆, as in “C₁-6 alkyl” is defined to include groups having 1, 2, 3, 4, 5, or 6 carbons in a linear or branched arrangement. For example, “C₁₋₈ alkyl” includes but is not limited to methyl, ethyl, n-propyl, i-propyl, n-butyl, t-butyl, i-butyl, pentyl, hexyl, heptyl, and octyl.

The term “cycloalkyl” means a saturated aliphatic cyclic hydrocarbon group having the specified number of carbon atoms. Unless otherwise specified, “cycloalkyl” refers to C₃₋₁₀ cycloalkyl. For example, “cycloalkyl” includes but is not limited to cyclopropyl, methyl-cyclopropyl, 2,2-dimethyl-cyclobutyl, 2-ethyl-cyclopentyl, and cyclohexyl.

The term “alkenyl” refers to a non-aromatic hydrocarbon radical, straight, branched or cyclic, containing from 2 to 10 carbon atoms and at least one carbon to carbon double bond. In some embodiments, one carbon to carbon double bond is present, and up to four non-aromatic carbon-carbon double bonds may be present. Thus, “C₂₋₆ alkenyl” means an alkenyl radical having from 2 to 6 carbon atoms. Alkenyl groups include but are not limited to ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. In some embodiments, up to three carbon-carbon triple bonds may be present. Thus, “C₂₋₆ alkynyl” means an alkynyl radical having from 2 to 6 carbon atoms. Alkynyl groups include but are not limited to ethynyl, propynyl, butyryl, and 3-methylbutynyl. The straight, branched or cyclic portion of the alkynyl group may contain triple bonds and may be substituted if a substituted alkynyl group is indicated.

The term “halogen” (or “halo”) refers to fluorine, chlorine, bromine and iodine.

The term “aryl” encompasses: 5- and 6-membered carbocyclic aromatic rings, for example, benzene; bicyclic ring systems wherein at least one ring is carbocyclic and aromatic, for example, naphthalene, indane, and 1, 2, 3, 4-tetrahydroquinoline; and tricyclic ring systems wherein at least one ring is carbocyclic and aromatic, for example, fluorene. In cases where the aryl substituent is bicyclic or tricyclic and at least one ring is non-aromatic, it is understood that attachment is via the aromatic ring.

For example, aryl includes 5- and 6-membered carbocyclic aromatic rings fused to a 5- to 7-membered heterocyclic ring containing one or more heteroatoms selected from N, O, and S, provided that the point of attachment is at the carbocyclic aromatic ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring, the resulting ring system is heteroaryl, not aryl, as defined herein.

The term “heteroaryl” refers to

5- to 8-membered aromatic, monocyclic rings containing one or more, for example, from 1 to 4, or, in some embodiments, from 1 to 3, heteroatoms selected from N, O, and S, with the remaining ring atoms being carbon;

8- to 12-membered bicyclic rings containing one or more, for example, from 1 to 4, or, in some embodiments, from 1 to 3, heteroatoms selected from N, O, and S, with the remaining ring atoms being carbon and wherein at least one heteroatom is present in an aromatic ring; and

11- to 14-membered tricyclic rings containing one or more, for example, from 1 to 4, or in some embodiments, from 1 to 3, heteroatoms selected from N, O, and S, with the remaining ring atoms being carbon and wherein at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 1-pyrazolyl, 2,3-pyrazolyl, 2,4-imidazolinyl, isoxazolyl, oxazolyl, thiazolyl, thiadiazolyl, tetrazolyl, thienyl, benzothienyl, furyl, benzofuryl, benzoimidazolinyl, indolinyl, pyridizinyl, triazolyl, quinolinyl, pyrazolyl, and 5,6,7,8-tetrahydroisoquinoline.

Further heteroaryl groups include but are not limited to pyrrolyl, isothiazolyl, triazinyl, pyrazinyl, pyridazinyl, indolyl, benzotriazolyl, quinoxalinyl, and isoquinolinyl. As with the definition of heterocycle below, “heteroaryl” is also understood to include the N-oxide derivative of any nitrogen-containing heteroaryl.

Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

In cases where the heteroaryl substituent is bicyclic or tricyclic and at least one ring is non-aromatic or contains no heteroatoms, it is understood that attachment is via the aromatic ring or via the heteroatom containing ring, respectively.

The term “heterocycle” (and variations thereof such as “heterocyclic”, or “heterocyclyl”) broadly refers to a single aliphatic ring, usually with 3 to 12 ring atoms, containing at least 2 carbon atoms in addition to one or more, preferably one to three heteroatoms independently selected from oxygen, sulfur, and nitrogen, as well as combinations comprising at least one of the foregoing heteroatoms. Alternatively, a heterocycle as defined above may be multicyclic ring system (e.g. bicyclic) in which two or more rings may be fused or bridged or spiro together, wherein at least one such ring contains one or more heteroatoms independently selected from oxygen, sulfur, and nitrogen. “Heterocycle” also refers to 5- to 7-membered heterocyclic ring containing one or more heteroatoms selected from N, O, and S fused with 5- and 6-membered carbocyclic aromatic ring, provided that the point of attachment is at the heterocyclic ring. The rings may be saturated or have one or more double bonds (i.e. partially unsaturated). The heterocycle can be substituted by oxo. The point of the attachment may be carbon or heteroatom in the heterocyclic ring, provided that attachment results in the creation of a stable structure. When the heterocyclic ring has substituents, it is understood that the substituents may be attached to any atom in the ring, whether a heteroatom or a carbon atom, provided that a stable chemical structure results. Heterocycle does not overlap with heteroaryl.

Suitable heterocycles include, but are not limited to, (as numbered from the linkage position assigned priority 1), 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, and 2,5-piperazinyl. Morpholinyl groups are also contemplated, including 2-morpholinyl and 3-morpholinyl (numbered wherein the oxygen is assigned priority 1). Substituted heterocycle also includes ring systems substituted with one or more oxo moieties, such as piperidinyl N-oxide, morpholinyl-N-oxide, 1-oxo-1-thiomorpholinyl and 1,1-dioxo-1-thiomorpholinyl. Bicyclic heterocycles include, but are not limited to,

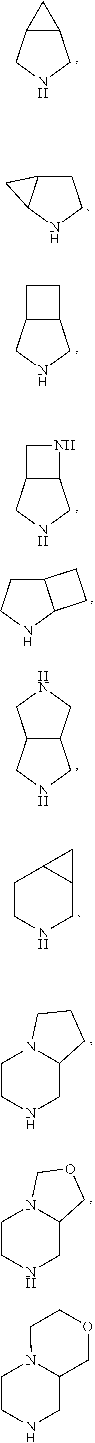

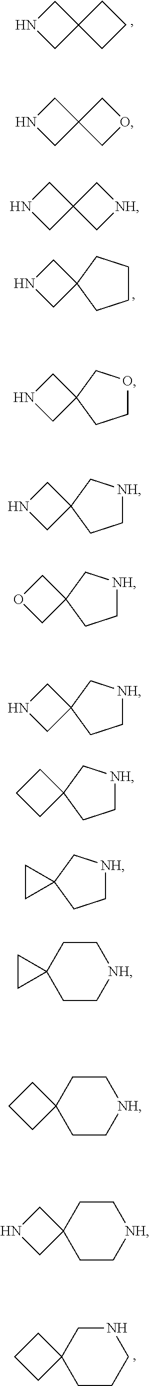

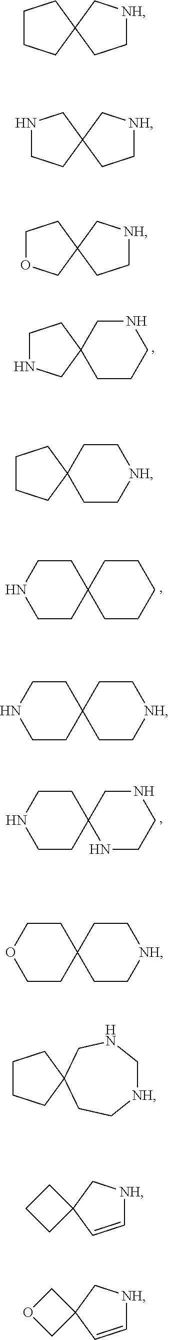

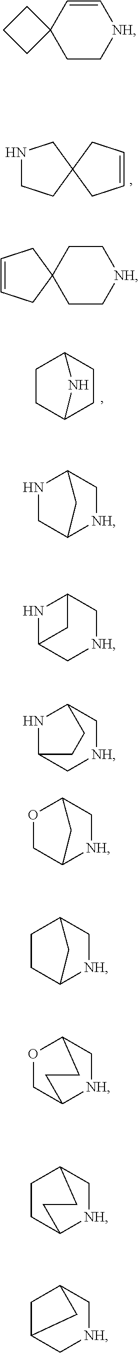

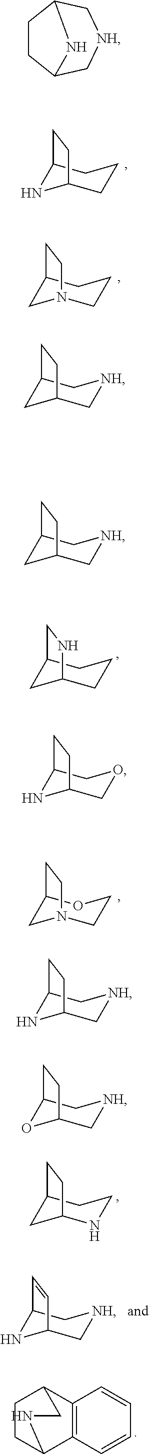

As used herein, “arylalkyl” refers to an alkyl moiety substituted by an aryl group. Example arylalkyl groups include benzyl, phenethyl, and naphthylmethyl groups. In some embodiments, arylalkyl groups have from 7 to 20 or 7 to 11 carbon atoms. When used in the phrase “arylC₁₋₄ alkyl”, the term “C₁₋₄” refers to the alkyl portion of the moiety and does not describe the number of atoms in the aryl portion of the moiety. Likewise, when used in the phrase “arylC₁₋₁₀ alkyl”, the term “C₁₋₁₀” refers to the alkyl portion of the moiety and does not describe the number of atoms in the aryl portion of the moiety.

As used herein, “heterocyclylalkyl” refers to alkyl substituted by heterocyclyl. When used in the phrase “heterocyclyl-C₁₋₆ alkyl”, the term “C₁₋₆” refers to the alkyl portion of the moiety and does not describe the number of atoms in the heterocyclyl portion of the moiety.

As used herein, “cycloalkylalkyl” refers to alkyl substituted by cycloalkyl. When used in the phrase “C₃₋₁₀ cycloalkylalkyl”, the term “C₃₋₁₀” refers to the cycloalkyl portion of the moiety and does not describe the number of atoms in the alkyl portion of the moiety. When used in the phrase “C₃₋₇ cycloalkylalkyl”, the term “C₃₋₇” refers to the cycloalkyl portion of the moiety and does not describe the number of atoms in the alkyl portion of the moiety. When used in the phrase “C₃₋₈ cycloalkylalkyl”, the term “C₃₋₈” refers to the cycloalkyl portion of the moiety and does not describe the number of atoms in the alkyl portion of the moiety. When used in the phrase “cycloalkyl C₁₋₁₀ alkyl”, the term “C₁₋₁₀” refers to the alkyl portion of the moiety and does not describe the number of atoms in the cycloalkyl portion of the moiety.

As used herein, “heteroarylalkyl” refers to alkyl substituted by heteroaryl. When used in the phrase “heteroaryl C₁₋₄ alkyl”, the term “C₁₋₄” refers to the alkyl portion of the moiety and does not describe the number of atoms in the heteroaryl portion of the moiety. Likewise, when used in the phrase “heteroaryl C₁₋₁₀ alkyl”, the term “C₁₋₁₀” refers to the alkyl portion of the moiety and does not describe the number of atoms in the heteroaryl portion of the moiety.

For avoidance of doubt, reference, for example, to substitution of alkyl, cycloalkyl, heterocyclyl, aryl, and/or heteroaryl refers to substitution of each of those groups individually as well as to substitutions of combinations of those groups. That is, if R¹ is arylalkyl, the aryl portion may be unsubstituted or substituted with at least one substituent, such as one, two, three, or four substituents, independently selected from R^{6b} and the alkyl portion may also be unsubstituted or substituted with at least one substituent, such as one, two, three, or four substituents, independently selected from R^{6a}.

The term “pharmaceutically acceptable salts” refers to salts prepared from pharmaceutically acceptable non-toxic bases or acids including inorganic or organic bases and inorganic or organic acids. Salts derived from inorganic bases may be selected, for example, from aluminum, ammonium, calcium, copper, ferric, ferrous, lithium, magnesium, manganic, manganous, potassium, sodium, and zinc salts. Further, for example, the pharmaceutically acceptable salts derived from inorganic bases may be selected from ammonium, calcium, magnesium, potassium, and sodium salts. Salts in the solid form may exist in one or more crystal structures, and may also be in the form of hydrates. Salts derived from pharmaceutically acceptable organic non-toxic bases may be selected, for example, from salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, and tripropylamine, tromethamine.

When the compound disclosed herein is basic, salts may be prepared using at least one pharmaceutically acceptable non-toxic acid, selected from inorganic and organic acids. Such acid may be selected, for example, from acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acids. In some embodiments, such acid may be selected, for example, from citric, hydrobromic, hydrochloric, maleic, phosphoric, sulfuric, fumaric, and tartaric acids.

The term “protecting group” or “Pg” refers to a substituent that can be commonly employed to block or protect a certain functionality while reacting other functional groups on the compound. For example, an “amino-protecting group” is a substituent attached to an amino group that blocks or protects the amino functionality in the compound. Suitable amino-protecting groups include but are not limited to acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include but are not limited to acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The terms “administration of” and or “administering” a compound and/or a pharmaceutically acceptable salt should be understood to mean providing a compound and/or a pharmaceutically acceptable salt thereof to the individual in recognized need of treatment.

The term “effective amount” means the amount of the a compound and/or a pharmaceutically acceptable salt that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to a pharmaceutical composition is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients.

The term “pharmaceutically acceptable” it is meant compatible with the other ingredients of the formulation and not unacceptably deleterious to the recipient thereof.

The term “subject” as used herein in reference to individuals suffering from a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

1. A compound of formula (I):

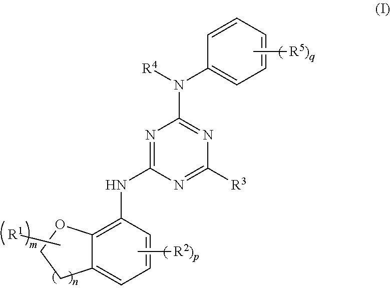

or a pharmaceutically acceptable salt thereof, wherein:

each R¹ is independently selected from hydrogen, halogen, hydroxyl, C₁₋₁₀ alkyl (such as C₁₋₆ alkyl), C₃₋₁₀ cycloalkyl (such as C₃₋₆ cycloalkyl), C₃₋₁₀ cycloalkyl-C₁₋₄ alkyl, heterocyclyl, heterocyclyl-C₁₋₄ alkyl, aryl, aryl-C₁₋₄ alkyl, heteroaryl, and heteroaryl-C₁₋₄ alkyl, wherein alkyl, cycloalkyl, and heterocyclyl are each unsubstituted or substituted with at least one substituent, such as one, two, three, or four substituents, independently selected from R^{6a}, and wherein aryl and heteroaryl are each unsubstituted or substituted with at least one substituent, such as one, two, three, or four substituents, independently selected from R^{6b};

each R² is independently selected from hydrogen, halogen, hydroxyl, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, heterocyclylcarbonyl, aryl, heteroaryl, aryl-C₁₋₄ alkyl, and heteroaryl-C₁₋₄ alkyl, wherein alkyl, alkenyl, alkynyl, cycloalkyl, and heterocyclyl are each unsubstituted or substituted with at least one substituent, such as one, two, three, or four substituents, independently selected from R^{6a}, and each aryl and heteroaryl is unsubstituted or substituted with at least one substituent, such as one, two, three, or four substituents, independently selected from R^{6b};

each R³ is independently selected from hydrogen, halogen, —CN, —NR⁷R⁸, and C₁₋₁₀ alkyl; wherein alkyl is unsubstituted or substituted with at least one substituent, such as one, two, three, or four substituents, independently selected from R^{6a};

each R⁴ is independently selected from hydrogen, halogen, —CN, C₁₋₁₀ alkyl, C₂₋₁₀ alkenyl, C₂₋₁₀ alkynyl, and C₃₋₁₀ cycloalkyl; wherein alkyl, alkenyl, alkynyl, and cycloalkyl are each unsubstituted or substituted with at least one substituent, such as one, two, three, or four substituents, independently selected from R^{6a};

or R³ and R⁴ together with the carbon atoms to which they are attached form a 5-6 membered ring containing 0, 1, 2 or 3 heteroatoms independently selected from oxygen, sulfur and nitrogen, and optionally substituted with 1-2 R^{6b} groups;

each R⁵ is independently selected from hydrogen, C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃; wherein alkyl, alkenyl, alkynyl, and cycloalkyl are each unsubstituted or substituted with at least one substituent, such as one, two, three, or four substituents, independently selected from R^{6a};

each R^{6a} is independently selected from C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —(CR⁹R¹⁰)ₜOR⁸, —(CR⁹R¹⁰)ₜNR⁷R⁸, —(CR⁹R¹⁰)ₜSR⁸, —(CR⁹R¹⁰)ₜS(O)ᵣR⁸, —(CR⁹R¹⁰)ₜCO₂R⁸, —(CR⁹R¹⁰)ₜCONR⁷R⁸, —(CR⁹R¹⁰)ₜNR⁷CO₂R⁸, —(CR⁹R¹⁰)ₜOCONR⁷R⁸, —(CR⁹R¹⁰)ₜNR⁷CONR⁷R⁸, —(CR⁹R¹⁰)ₜNR⁷SO₂NR⁷R⁸, —O(CR⁹R¹⁰)ₜNR⁷R⁸, —C(O)R⁷, —C(O)(CR⁹R¹⁰)ₜOR⁸, —C(O)(CR⁹R¹⁰)ₜNR⁷R⁸, —C(O)(CR⁹R¹⁰)ₜSR⁸, —C(O)(CR⁹R¹⁰)ₜS(O)ᵣR⁸, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃;

each R^{6b} is independently selected from R^{6a}, aryl, aryl-C₁₋₄ alkyl, heteroaryl, and heteroaryl-C₁₋₄ alkyl;

each R⁷ and each R⁸ are independently selected from hydrogen, C₁₋₁₀ alkyl, C₂₋₁₀ alkenyl, C₂₋₁₀ alkynyl, cycloalkyl, cycloalkyl-C₁₋₄ alkyl; heterocyclyl, heterocyclyl-C₁₋₄ alkyl, aryl, heteroaryl, aryl-C₁₋₄ alkyl, and heteroaryl-C₁₋₄ alkyl; wherein alkyl, alkenyl, alkynyl, cycloalkyl, and heterocyclyl are each unsubstituted or substituted with at least one substituent, such as one, two, three, or four substituents, independently selected from R^{6a}, and aryl and heteroaryl are each unsubstituted or substituted with at least one substituent, such as one, two, three, or four substituents, independently selected from R^{6b}; or

R⁷ and R⁸ together with the atom(s) to which they are attached form a heterocyclic ring of 4 to 7 members containing 0, 1, or 2 additional heteroatoms independently selected from oxygen, sulfur and NR¹¹, each R⁷ and R⁸ may be unsubstituted or substituted on a carbon or nitrogen atom with at least one substituent, such as one, two, or three substituents, selected from R¹²;

each R⁹ and each R¹⁰ are independently selected from: hydrogen, C₁₋₁₀ alkyl, C₂₋₁₀ alkenyl, C₂₋₁₀ alkynyl, cycloalkyl, cycloalkyl-C₁₋₄ alkyl, heterocyclyl, heterocyclyl-C₁₋₄ alkyl, aryl, heteroaryl, aryl-C₁₋₄ alkyl, and heteroaryl-C₁₋₄ alkyl; or

R⁹ and R¹⁰ together with the carbon atom(s) to which they are attached form a ring of 3 to 7 members containing 0, 1, or 2 heteroatoms independently selected from oxygen, sulfur and nitrogen, and optionally substituted with 1-2 R^{6a} groups;

each R¹¹ is independently selected from hydrogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —S(O)ᵣR⁷, —C(O)R⁷, —CO₂R⁷, —CO₂(CR⁹R¹⁰)ₜCONR⁷R⁸, and —C(O)NR⁷R⁸;

each R¹² is independently selected from halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —OR⁷, —NR⁷S(O)ᵣR⁸, —S(O)ᵣR⁷, —SR⁷, —S(O)₂OR⁷, —OS(O)₂R⁷, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CHF₂, —CF₃, —OCHF₂, and —OCF₃;

each m is independently selected from 0, 1 and 2;

each n is independently selected from 1, 2, and 3;

each p is independently selected from 0, 1, 2, and 3;

each q is independently selected from 0, 1, 2, and 3;

each r is independently selected from 1 and 2;

each t is independently selected from 1, 2, and 3.

2. A compound of 1 or a pharmaceutically acceptable salt thereof, wherein R² is selected from C₁₋₁₀ alkyl and heterocyclyl, wherein alkyl and heterocyclyl are each unsubstituted or substituted with at least one substituent, such as one, two, three, or four substituents, independently selected from R^{6a}, and R^{6a} is described as in 1.

3. A compound of 2 or a pharmaceutically acceptable salt thereof, wherein R² is selected from methyl and piperidinyl, wherein piperidinyl is unsubstituted or substituted with at least one substituent, such as one, two, three, or four substituents, independently selected from R^{6a}, and R^{6a} is described as in 1.

4. A compound of 3 or a pharmaceutically acceptable salt thereof, wherein the substructure of Formula (II):

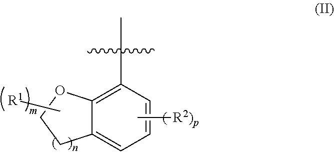

in Formula (I) is selected from

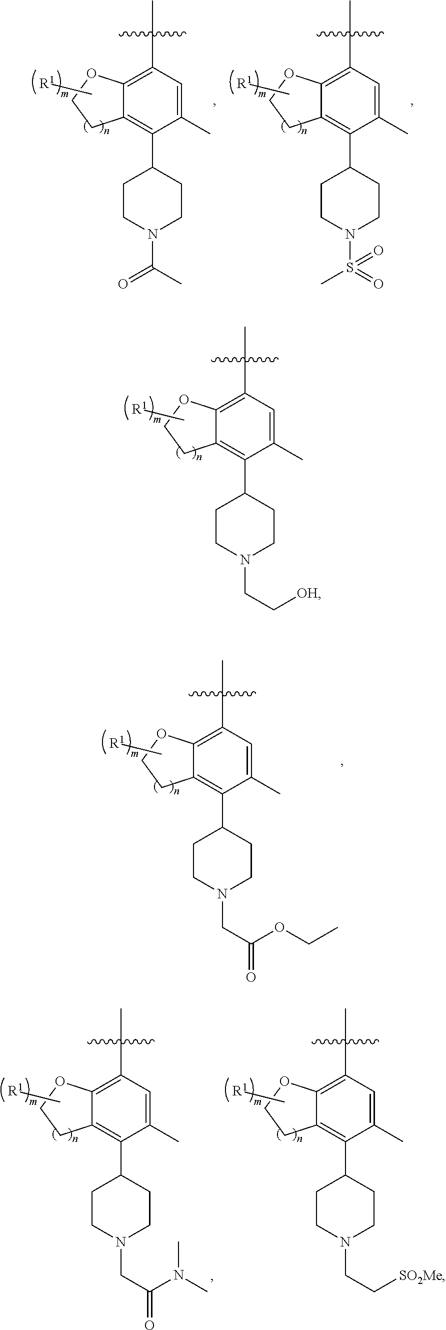

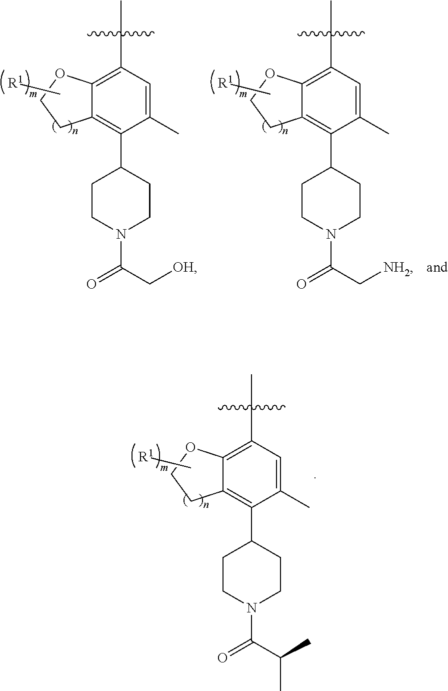

5. A compound of 1 or a pharmaceutically acceptable salt thereof, wherein R¹ is C₁₋₁₀ alkyl.

6. A compound of 5 or a pharmaceutically acceptable salt thereof, wherein R¹ is methyl and m=1.

7. A compound of 1 or a pharmaceutically acceptable salt thereof, wherein R⁵ is —S(O)ᵣR⁷, wherein R⁷ and r are described as in 1.

8. A compound of 7 or a pharmaceutically acceptable salt thereof, wherein substructure of Formula (III):

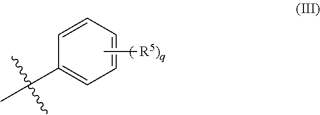

### in Formula (I) is

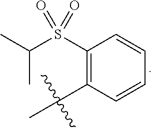 [SMILES: *c1ccccc1S(=O)(=O)C(C)C]

9. A compound of 1 or a pharmaceutically acceptable salt thereof, wherein R³ is hydrogen.

10. A compound of 1 or a pharmaceutically acceptable salt thereof, wherein R⁴ is hydrogen.

11. A compound of 1 or a pharmaceutically acceptable salt thereof, wherein n is 1.

12. A compound, selected from
(R)—N²-(2,5-dimethyl-4-(piperidin-4-yl)-2,3-dihydrobenzofuran-7-yl)-N⁴-(2-(isopropylsulfonyl)phenyl)-1,3,5-triazine-2,4-diamine,
(R)—N²-(2,5-dimethyl-4-(1-methylpiperidin-4-yl)-2,3-dihydrobenzofuran-7-yl)-N⁴-(2-(isopropylsulfonyl)phenyl)-1,3,5-triazine-2,4-diamine,
(R)-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one
(R)—N²-(2,5-dimethyl-4-(1-(methylsulfonyl)piperidin-4-yl)-2,3-dihydrobenzofuran-7-yl)-N⁴-(2-(isopropylsulfonyl)phenyl)-1,3,5-triazine-2,4-diamine,
(R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-ol,
ethyl (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)acetate,
(R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-N,N-dimethylacetamide,
(R)—N²-(2,5-dimethyl-4-(1-(2-(methylsulfonyl)ethyl)piperidin-4-yl)-2,3-dihydrobenzofuran-7-yl)-N⁴-(2-(isopropylsulfonyl)phenyl)-1,3,5-triazine-2,4-diamine,
(R)-2-hydroxy-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one,
(R)-2-amino-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one,
(S)-2-amino-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)propan-1-one,
   or pharmaceutically acceptable salt thereof.

In another of its aspects, there is provided a pharmaceutical composition comprising a compound according to any one of 1-12, and/or a pharmaceutically acceptable salts thereof, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another of its aspects, there is provided a kit comprising a compound of any one of 1-12, and/or a pharmaceutically acceptable salts thereof; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound of any one of 1-12, and/or a pharmaceutically acceptable salts thereof and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound of 1-12, and/or a pharmaceutically acceptable salts thereof to a subject.

In another of its aspects, there is provided a method of inhibiting ALK kinase comprising contacting the ALK with a compound of any one of 1-12, and/or a pharmaceutically acceptable salts thereof.

In yet another of its aspects, there is provided a method of inhibiting a ALK comprising causing a compound of any one of 1-12, and/or a pharmaceutically acceptable salts thereof, to be present in a subject in order to inhibit the ALK in vivo.

In a further of its aspects, there is provided a method of inhibiting ALK comprising administering a first compound to a subject that is converted in vivo to a second compound wherein the second compound inhibits the ALK in vivo, the second compound being a compound according to any one of 1-12, or a pharmaceutically acceptable salts thereof.

In another of its aspects, there is provided a method of treating a disease state for which a ALK possesses activity that contributes to the pathology and/or symptomology of the disease state, the method comprising causing a compound of any one of 1-12, or a pharmaceutically acceptable salts thereof, to be present in a subject in a therapeutically effective amount for the disease state.

In a further of its aspects, there is provided a method of treating a disease state for which a ALK possesses activity that contributes to the pathology or symptomology of the disease state, the method comprising administering a first compound to a subject that is converted in vivo to a second compound wherein the second compound inhibits the ALK in vivo. It is noted that the compounds of the present invention may be the first or second compounds.

In one variation of each of the above methods the disease state is selected from the group consisting of cancerous hyperproliferative disorders (e.g., brain, lung, squamous cell, bladder, gastric, pancreatic, breast, head, neck, renal, kidney, ovarian, prostate, colorectal, epidermoid, esophageal, testicular, gynecological or thyroid cancer); non-cancerous hyperproliferative disorders (e.g., benign hyperplasia of the skin (e.g., psoriasis), restenosis, and benign prostatic hypertrophy (BPH)); pancreatitis; kidney disease; pain; preventing blastocyte implantation; treating diseases related to vasculogenesis or angiogenesis (e.g., tumor angiogenesis, acute and chronic inflammatory disease such as rheumatoid arthritis, atherosclerosis, inflammatory bowel disease, skin diseases such as psoriasis, exzema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer); asthma; neutrophil chemotaxis (e.g., reperfusion injury in myocardial infarction and stroke and inflammatory arthritis); septic shock; T-cell mediated diseases where immune suppression would be of value (e.g., the prevention of organ transplant rejection, graft versus host disease, lupus erythematosus, multiple sclerosis, and rheumatoid arthritis); atherosclerosis; inhibition of keratinocyte responses to growth factor cocktails; chronic obstructive pulmonary disease (COPD) and other diseases.

In another of its aspects, there is provided a method of treating a disease state for which a mutation in the ALK gene contributes to the pathology and/or symptomology of the disease state including, for example, melanomas, lung cancer, colon cancer and other tumor types.

In still another of its aspects, the present invention relates to the use of a compound of any of the above embodiments and variations as a medicament. In yet another of its aspects, the present invention relates to the use of a compound according to any one of 1-12, and/or a pharmaceutically acceptable salts thereof, in the manufacture of a medicament for inhibiting an ALK.

In a further of its aspects, the present invention relates to the use of a compound according to any one of 1-12, or a pharmaceutically acceptable salts thereof, in the manufacture of a medicament for treating a disease state for which an ALK possesses activity that contributes to the pathology or symptomology of the disease state.

### Administration and Pharmaceutical Compositions

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors known to those of ordinary skill in the art. For example, for the treatment of neoplastic diseases and immune system disorders, the required dosage will also vary depending on the mode of administration, the particular condition to be treated and the effect desired.

In general, satisfactory results are indicated to be obtained systemically at daily dosages of from about 0.001 to about 100 mg/kg per body weight, or particularly, from about 0.03 to 2.5 mg/kg per body weight. An indicated daily dosage in the larger mammal, e.g. humans, may be in the range from about 0.5 mg to about 2000 mg, or more particularly, from about 0.5 mg to about 1000 mg, conveniently administered, for example, in divided doses up to four times a day or in retard form. Suitable unit dosage forms for oral administration comprise from ca. 1 to 50 mg active ingredient.

Compounds of the disclosure may be administered as pharmaceutical compositions by any conventional route; for example, enterally, e.g., orally, e.g., in the form of tablets or capsules; parenterally, e.g., in the form of injectable solutions or suspensions; or topically, e.g., in the form of lotions, gels, ointments or creams, or in a nasal or suppository form.

Pharmaceutical compositions comprising a compound of the present disclosure in free form or in a pharmaceutically acceptable salt form in association with at least one pharmaceutically acceptable carrier or diluent may be manufactured in a conventional manner by mixing, granulating, coating, dissolving or lyophilizing processes. For example, pharmaceutical compositions comprising a compound of the disclosure in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

In one embodiment, the pharmaceutical compositions are solutions of the active ingredient, including suspensions or dispersions, such as isotonic aqueous solutions. In the case of lyophilized compositions comprising the active ingredient alone or together with a carrier such as mannitol, dispersions or suspensions can be made up before use. The pharmaceutical compositions may be sterilized and/or contain adjuvants, such as preserving, stabilizing, wetting or emulsifying agents, solution promoters, salts for regulating the osmotic pressure and/or buffers. Suitable preservatives include but are not limited to antioxidants such as ascorbic acid, or microbicides, such as sorbic acid or benzoic acid. The solutions or suspensions may further comprise viscosity-increasing agents, including but not limited to, sodium carboxymethylcellulose, carboxymethylcellulose, dextran, polyvinylpyrrolidone, gelatins, or solubilizers, e.g. Tween 80 (polyoxyethylene(20)sorbitan mono-oleate).

Suspensions in oil may comprise as the oil component the vegetable, synthetic, or semi-synthetic oils customary for injection purposes. Examples include liquid fatty acid esters that contain as the acid component a long-chained fatty acid having from 8 to 22 carbon atoms, or in some embodiments, from 12 to 22 carbon atoms. Suitable liquid fatty acid esters include but are not limited to lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid and linoleic acid, and if desired, may contain antioxidants, for example vitamin E, 3-carotene or 3,5-di-tert-butyl-hydroxytoluene. The alcohol component of these fatty acid esters may have six carbon atoms and may be monovalent or polyvalent, for example a mono-, di- or trivalent, alcohol. Suitable alcohol components include but are not limited to methanol, ethanol, propanol, butanol or pentanol or isomers thereof; glycol and glycerol.

Other suitable fatty acid esters include but are not limited ethyl-oleate, isopropyl myristate, isopropyl palmitate, LABRAFIL® M 2375, (polyoxyethylene glycerol), LABRAFIL® M 1944 CS (unsaturated polyglycolized glycerides prepared by alcoholysis of apricot kernel oil and comprising glycerides and polyethylene glycol ester), LABRASOL™ (saturated polyglycolized glycerides prepared by alcoholysis of TCM and comprising glycerides and polyethylene glycol ester; all available from GaKefosse, France), and/or MIGLYOL® 812 (triglyceride of saturated fatty acids of chain length C₈ to C₁₂ from Hüls AG, Germany), and vegetable oils such as cottonseed oil, almond oil, olive oil, castor oil, sesame oil, soybean oil, or groundnut oil.

Pharmaceutical compositions for oral administration may be obtained, for example, by combining the active ingredient with one or more solid carriers, and if desired, granulating a resulting mixture, and processing the mixture or granules by the inclusion of additional excipients, to form tablets or tablet cores.

Suitable carriers as used herein, refers to relatively nontoxic chemical compounds or agents that facilitate the incorporation of a compound into cells or tissues, which include but are not limited to fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, carboxymethyl starch, crosslinked polyvinylpyrrolidone, alginic acid or a salt thereof, such as sodium alginate. Additional excipients include flow conditioners and lubricants, for example silicic acid, talc, stearic acid or salts thereof, such as magnesium or calcium stearate, and/or polyethylene glycol, or derivatives thereof.

Tablet cores may be provided with suitable, optionally enteric, coatings through the use of, inter alia, concentrated sugar solutions which may comprise gum arable, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different doses of active ingredient.

Pharmaceutical compositions for oral administration may also include hard capsules comprising gelatin or soft-sealed capsules comprising gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient may be dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories comprising a combination of the active ingredient and a suppository base. Suitable suppository bases are, for example, natural or synthetic triglycerides, paraffin hydrocarbons, polyethylene glycols or higher alkanols.

Pharmaceutical compositions suitable for parenteral administration may comprise aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers. The active ingredient, optionally together with excipients, can also be in the form of a lyophilizate and can be made into a solution before parenteral administration by the addition of suitable solvents. Solutions such as are used, for example, for parenteral administration can also be employed as infusion solutions. The manufacture of injectable preparations is usually carried out under sterile conditions, as is the filling, for example, into ampoules or vials, and the sealing of the containers.

The compounds of the disclosure may be administered as the sole active ingredient, or together with other drugs useful against neoplastic diseases or useful in immunomodulating regimens. For example, the compounds of the disclosure may be used in accordance with the disclosure in combination with pharmaceutical compositions effective in various diseases as described above, e.g. with cyclophosphamide, 5-fluorouracil, fludarabine, gemcitabine, cisplatinum, carboplatin, vincristine, vinblastine, etoposide, irinotecan, paclitaxel, docetaxel, rituxan, doxorubicine, gefitinib, or imatinib; or also with cyclosporins, rapamycins, ascomycins or their immunosuppressive analogs, e.g. cyclosporin A, cyclosporin G, FK-506, sirolimus or everolimus, corticosteroids, e.g. prednisone, cyclophosphamide, azathioprene, methotrexate, gold salts, sulfasalazine, antimalarials, brequinar, leflunomide, mizoribine, mycophenolic acid, mycophenolate, mofetil, 15-deoxyspergualine, immuno-suppressive monoclonal antibodies, e.g. monoclonal antibodies to leukocyte receptors, e.g. MHC, CD2, CD3, CD4, CD7, CD25, CD28, I CD40, CD45, CD58, CD80, CD86, CD152, CD137, CD154, ICOS, LFA-1, VLA-4 or their ligands, or other immunomodulatory compounds, e.g. CTLA41g.

The disclosure also provides for a pharmaceutical combinations, e.g. a kit, comprising a) a first agent which is a compound of the disclosure as disclosed herein, in free form or in pharmaceutically acceptable salt form, and b) at least one co-agent. The kit can comprise instructions for its administration.

### EXAMPLES

Various methods may be developed for synthesizing a compound of formula (I) or a pharmaceutically acceptable salt thereof. Representative methods for synthesizing a compound of formula (I) and/or a pharmaceutically acceptable salt thereof are provided in the Examples. It is noted, however, that a compound of formula (I) or a pharmaceutically acceptable salt thereof may also be synthesized by other synthetic routes that others may devise.

It will be readily recognized that certain compounds of formula (I) have atoms with linkages to other atoms that confer a particular stereochemistry to the compound (e.g., chiral centers). It is recognized that synthesis of a compound of formula (I) and/or a pharmaceutically acceptable salt thereof may result in the creation of mixtures of different stereoisomers (enantiomers, diastereomers). Unless a particular stereochemistry is specified, recitation of a compound is intended to encompass all of the different possible stereoisomers.

A compound of formula (I) can also be prepared as a pharmaceutically acceptable acid addition salt by, for example, reacting the free base form of a compound with a pharmaceutically acceptable inorganic or organic acid. Alternatively, a pharmaceutically acceptable base addition salt of a compound of formula (I) can be prepared by, for example, reacting the free acid form of a compound with a pharmaceutically acceptable inorganic or organic base. Inorganic and organic acids and bases suitable for the preparation of the pharmaceutically acceptable salts of compounds of formula (I) are set forth in the definitions section of this Application. Alternatively, the salt forms of the compounds of formula (I) can be prepared using salts of the starting materials or intermediates.

The free acid or free base forms of the compounds of formula (I) can be prepared from the corresponding base addition salt or acid addition salt form. For example, a compound of formula (I) in an acid addition salt form can be converted to the corresponding free base thereof by treating with a suitable base (e.g., ammonium hydroxide solution, sodium hydroxide, and the like). A compound of formula (I) in a base addition salt form can be converted to the corresponding free acid thereof by, for example, treating with a suitable acid (e.g., hydrochloric acid, etc).

The N-oxides of a compound of formula (I) and/or a pharmaceutically acceptable salt thereof can be prepared by methods known to those of ordinary skill in the art. For example, N-oxides can be prepared by treating an unoxidized form of the compound of formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0 to 80° C. Alternatively, the N-oxides of the compounds of formula (I) can be prepared from the N-oxide of an appropriate starting material.

Compounds of formula (I) in an unoxidized form can be prepared from N-oxides of compounds of formula (I) by, for example, treating with a reducing agent (e.g., sulfur, sulfur dioxide, triphenyl phosphine, lithium borohydride, sodium borohydride, phosphorus trichloride, tribromide, and the like) in an suitable inert organic solvent (e.g., acetonitrile, ethanol, aqueous dioxane, and the like) at 0 to 80° C.

Protected derivatives of the compounds of formula (I) can be made by methods known to those of ordinary skill in the art. A detailed description of the techniques applicable to the creation of protecting groups and their removal can be found in T. W. Greene, Protecting Groups in Organic Synthesis, 3rd edition, John Wiley & Sons, Inc. 1999.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. For example, the following abbreviations may be used in the examples and throughout the specification: g (grams); mg (milligrams); L (liters); mL (milliliters); μL (microliters); psi (pounds per square inch); M (molar); mM (millimolar); i.v. (intravenous); Hz (Hertz); MHz (megahertz); mol (moles); mmol (millimoles); RT (room temperature); min (minutes); h (hours); mp (melting point); TLC (thin layer chromatography); Rt (retention time); RP (reverse phase); MeOH (methanol); i-PrOH (isopropanol); TEA (triethylamine); TFA (trifluoroacetic acid); TFAA (trifluoroacetic anhydride); THF (tetrahydrofuran); DMSO (dimethyl sulfoxide); EtOAc (ethyl acetate); DME (1,2-dimethoxyethane); DCM (dichloromethane); DCE (dichloroethane); DMF (N,N-dimethylformamide); DMPU (N,N′-dimethylpropyleneurea); CDI (1,1-carbonyldiimidazole); IBCF (isobutyl chloroformate); HOAc (acetic acid); HOSu (N-hydroxysuccinimide); HOBT (1-hydroxybenzotriazole); Et₂O (diethyl ether); EDCI (1-(3-dimethylaminopropyl)-3-ethylcarbodiimide hydrochloride); BOC (tert-butyloxycarbonyl); FMOC (9-fluorenylmethoxycarbonyl); DCC (dicyclohexylcarbodiimide); CBZ (benzyloxycarbonyl); Ac (acetyl); atm (atmosphere); TMSE (2-(trimethylsilyl)ethyl); TMS (trimethylsilyl); TIPS (triisopropylsilyl); TBS (t-butyldimethylsilyl); DMAP (4-dimethylaminopyridine); Me (methyl); OMe (methoxy); Et (ethyl); tBu (tert-butyl); HPLC (high pressure liquid chomatography); BOP (bis(2-oxo-3-oxazolidinyl)phosphinic chloride); TBAF (tetra-n-butylammonium fluoride); m-CPBA (meta-chloroperbenzoic acid).

References to ether or Et₂O are to diethyl ether; brine refers to a saturated aqueous solution of NaCl. Unless otherwise indicated, all temperatures are expressed in ° C. (degrees Centigrade). All reactions were conducted under an inert atmosphere at RT unless otherwise noted.

¹H NMR spectra were recorded on a Varian Mercury Plus 400. Chemical shifts are expressed in parts per million (ppm). Coupling constants are in units of hertz (Hz). Splitting patterns describe apparent multiplicities and are designated as s (singlet), d (doublet), t (triplet), q (quartet), m (multiplet), and br (broad).

Low-resolution mass spectra (MS) and compound purity data were acquired on a Shimadzu LC/MS single quadrapole system equipped with electrospray ionization (ESI) source, UV detector (220 and 254 nm), and evaporative light scattering detector (ELSD). Thin-layer chromatography was performed on 0.25 mm Superchemgroup silica gel plates (60F-254), visualized with UV light, 5% ethanolic phosphomolybdic acid, ninhydrin, or p-anisaldehyde solution. Flash column chromatography was performed on silica gel (200-300 mesh, Branch of Qingdao Haiyang Chemical Co., Ltd).

### Synthetic Schemes

A compound of formula I and/or a pharmaceutically acceptable salt thereof may be synthesized according to a variety of reaction schemes. Some illustrative schemes are provided below and in the examples. Other reaction schemes could be readily devised by those skilled in the art in view of the present disclosure.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

Synthetic methods for preparing the compounds of the present disclosure are illustrated in the following Schemes and Examples. Starting materials are commercially available or may be made according to procedures known in the art or as illustrated herein.

The intermediates shown in the following schemes are either known in the literature or may be prepared by a variety of methods familiar to those skilled in the art.

As shown in the Scheme 1, the compounds of formula I can be synthesized from analine II, dihalotriazine III and analine IV which are either known in the literature or may be prepared by a variety of methods familiar to those skilled in the art.

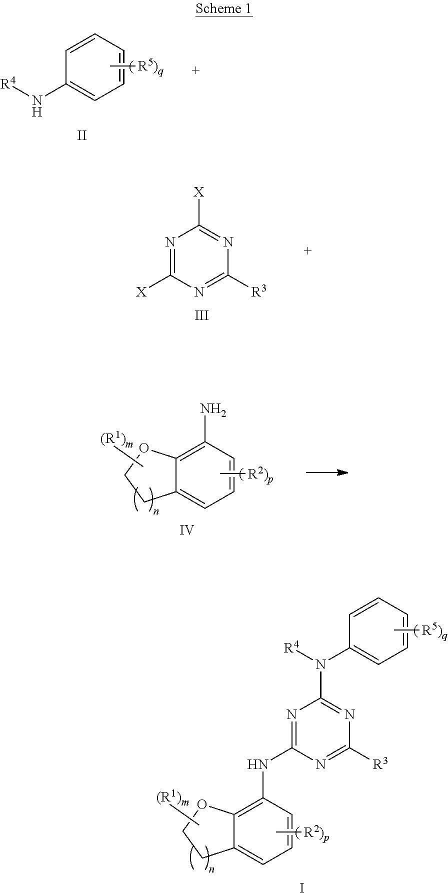

As an illustration of the preparation of compounds of formula I. One synthetic route is shown in Scheme 2. The preparation starts with analine II, which is commercially available or can be synthesized following the procedure known in the literature. Coupling of analine II with dihalotriazine III in the presence of a base such as DIPEA and in a solvent such as THF leads to intermediate V. Further reaction of intermediate V with analine IV in the presence of an acid such as TFA and in a solvent such as i-PrOH gives compounds of formula I. The coupling of analine II with dihalotrazine III and the coupling of intermediate V with analine IV may be effected by other known methods such as Buchwald reaction.

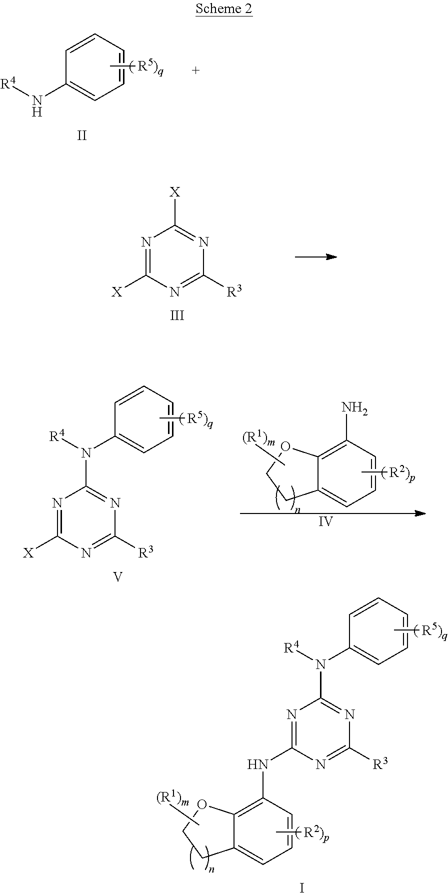

In some cases, compounds of formula I may be modified to give more derivatives of compound formula I by derivatization reactions. These derivatization reactions may include, but are not limited to, reductive amination, alkylation, acylation, condensation, substitution, reduction and oxidation reactions that are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Example 1

### (R)—N²-(2,5-dimethyl-4-(piperidin-4-yl)-2,3-dihydrobenzofuran-7-yl)-N⁴-(2-(isopropylsulfonyl)phenyl)-1,3,5-triazine-2,4-diamine (1)

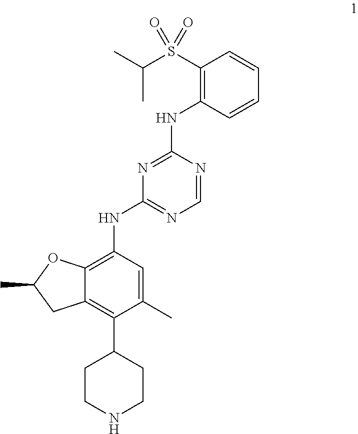 [SMILES: Cc1cc(Nc2ncnc(Nc3ccccc3S(=O)(=O)C(C)C)n2)c2c(c1C1CCNCC1)C[C@@H](C)O2]

### 4-chloro-N-(2-(isopropylsulfonyl)phenyl)-1,3,5-triazin-2-amine (1a)

To a solution of 2,4-dichloro-1,3,5-triazine (3 g, 20.1 mmol) in anhydrous THF (25 mL), a mixture of 2-(isopropylsulfonyl)aniline (1.76 g, 8.8 mmol), DIPEA (2.7 mL) and anhydrous THF (20 mL) was added dropwise, followed by stirring at room temperature for 60 h. The solvent was removed and the mixture was extracted with ethyl acetate (2×100 mL). The extracts were washed with saturated NaHCO₃ (50 mL), water, brine and dried over Na₂SO₄. The residue was purified by silica gel column eluting with PE:EtOAc (10:1 to 3:1) to give the title compound 4-chloro-N-(2-(isopropylsulfonyl)phenyl)-1,3,5-triazin-2-amine (1a). MS-ESI (m/z): 313 [M+1]⁺.

### (R)-2,5-dimethyl-4-(piperidin-4-yl)-2,3-dihydrobenzofuran-7-amine (1b)

(R)-2,5-dimethyl-4-(piperidin-4-yl)-2,3-dihydrobenzofuran-7-amine (1b) was prepared according to the method described in WO2014071832.

### (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1)

To a solution of (R)-2,5-dimethyl-4-(piperidin-4-yl)-2,3-dihydrobenzofuran-7-amine (1b) (304 mg, 1.24 mmol) in i-PrOH (15 mL) was added TFA (483 mg, 4.23 mmol). The mixture was stirred at room temperature for 5 min and then added 1a (330 mg, 1.06 mmol). The reaction mixture was heated to 50° C. for 4 h. The solvent was removed. The mixture was added 2 N HCl (6 mL) and extracted with ethyl acetate (50 mL). The aqueous layer was adjusted with 2.5 N NaOH to pH=9 and extracted with ethyl acetate (2×50 mL). The extracts were washed with brine and dried over Na₂SO₄. Solvents were evaporated under reduced pressure to give the title compound (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1). MS-ESI (m/z): 523 [M+1]⁺.

### Example 2

### (R)—N²-(2,5-dimethyl-4-(1-methylpiperidin-4-yl)-2,3-dihydrobenzofuran-7-yl)-N⁴-(2-(isopropyl-sulfonyl)phenyl)-1,3,5-triazine-2,4-diamine (2)

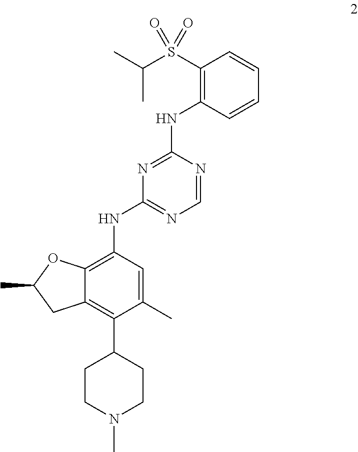 [SMILES: Cc1cc(Nc2ncnc(Nc3ccccc3S(=O)(=O)C(C)C)n2)c2c(c1C1CCN(C)CC1)C[C@@H](C)O2]

### (R)-2,5-dimethyl-4-(1-methylpiperidin-4-yl)-2,3-dihydrobenzofuran-7-amine

To a solution of (R)-2,5-dimethyl-4-(piperidin-4-yl)-2,3-dihydrobenzofuran-7-amine (1d) (14 mg, 0.06 mmol) in THF (2 mL) at room temperature was added formaldehyde (37% in water, 21 mg, 0.27 mmol), followed by NaBH(OAc)₃ (75 mg, 0.35 mmol). The mixture was stirred at room temperature overnight. The mixture was diluted with saturated NaHCO₃ (10 mL) and extracted with ethyl acetate (2×20 mL). The extracts were washed with water, brine and dried over Na₂SO₄. Solvents were evaporated under reduced pressure to give the crude product of the title compound (R)-2,5-dimethyl-4-(1-methylpiperidin-4-yl)-2,3-dihydrobenzofuran-7-amine (2a). MS-ESI (m/z): 261 [M+1]⁺.

### (R)—N²-(2,5-dimethyl-4-(1-methylpiperidin-4-yl)-2,3-dihydrobenzofuran-7-yl)-N⁴-(2-(isopropylsulfonyl)phenyl)-1,3,5-triazine-2,4-diamine (2)

To a solution of (R)-2,5-dimethyl-4-(1-methylpiperidin-4-yl)-2,3-dihydrobenzofuran-7-amine (2a) (29 mg, 0.11 mmol) in i-PrOH (5 mL) was added TFA (12 mg, 0.11 mmol) and 4-chloro-N-(2-(isopropylsulfonyl)phenyl)-1,3,5-triazin-2-amine (1a) (31 mg, 0.1 mmol). The reaction mixture was heated to 50° C. for 8 h. The solvent was removed and the mixture was extracted with ethyl acetate (2×30 mL). The extracts were washed with saturated NaHCO₃ (10 mL), water, brine and dried over Na₂SO₄. Solvents were evaporated under reduced pressure. The residue was purified by preparative thin layer chromatography eluting with DCM:MeOH (10:1) to give the title compound (R)—N²-(2,5-dimethyl-4-(1-methylpiperidin-4-yl)-2,3-dihydrobenzofuran-7-yl)-N⁴-(2-(isopropylsulfonyl)phenyl)-1,3,5-tri azine-2,4-diamine (2). MS-ESI (m/z): 537 [M+1]⁺.

### Example 3

### (R)-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (3)

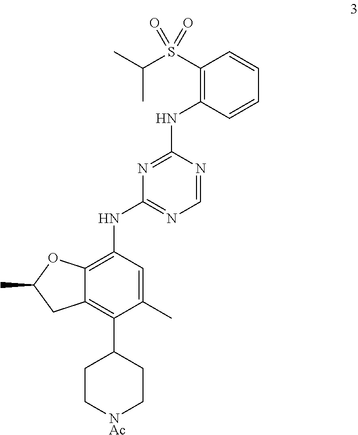

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (20 mg, 0.038 mmol) in anhydrous DCM (2 mL) was added Et₃N (32 μL, 0.23 mmol) and acetyl chloride (10 μL, 0.15 mmol) at 0° C. The mixture was warmed up to room temperature and stirred for 2 h. The mixture was diluted with water and extracted with DCM (2×30 mL). The extracts were washed with brine and dried over Na₂SO₄. Solvents were evaporated under reduced pressure. The residue was purified by preparative thin layer chromatography eluting with DCM:MeOH (30:1) to give the title compound (R)-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (3). MS-ESI (m/z): 565 [M+1]⁺.

### Example 4

### (R)—N²-(2,5-dimethyl-4-(1-(methylsulfonyl)piperidin-4-yl)-2,3-dihydrobenzofuran-7-yl)-N⁴-(2-(isopropylsulfonyl)phenyl)-1,3,5-triazine-2,4-diamine (4)

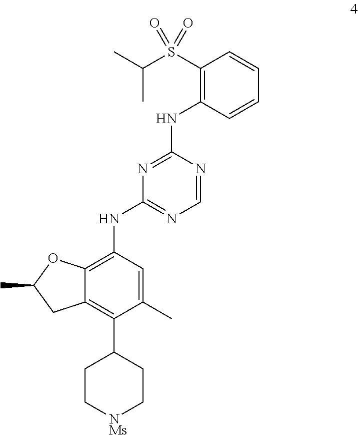

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (20 mg, 0.038 mmol) in anhydrous DCM (2 mL) was added Et₃N (32 μL, 0.23 mmol) and methanesulfonyl chloride (12 μL, 0.14 mmol) at 0° C. The mixture was warmed up to room temperature and stirred for 1.5 h. The mixture was diluted with water and extracted with DCM (2×30 mL). The extracts were washed with brine and dried over Na₂SO₄. Solvents were evaporated under reduced pressure. The residue was purified by preparative thin layer chromatography eluting with DCM:MeOH (30:1) to give the title compound (R)—N²-(2,5-dimethyl-4-(1-(methylsulfonyl)piperidin-4-yl)-2,3-dihydrobenzofuran-7-yl)-N⁴-(2-(isopropylsulfonyl)phenyl)-1,3,5-triazine-2,4-diamine (4). MS-ESI (m/z): 601 [M+1]⁺.

### Example 5

### (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-ol (5)

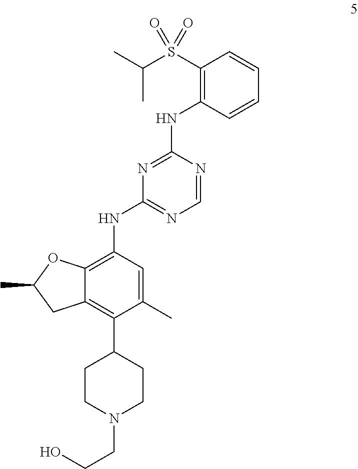 [SMILES: Cc1cc(Nc2ncnc(Nc3ccccc3S(=O)(=O)C(C)C)n2)c2c(c1C1CCN(CCO)CC1)C[C@@H](C)O2]

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (30 mg, 0.057 mmol) in anhydrous DMF (1.5 mL) was added Et₃N (20 μL, 0.14 mmol) and 2-bromoethan-1-ol (10 mg, 0.06 mmol). The reaction mixture was heated to 50° C. for 6 h. The mixture was extracted with ethyl acetate (2×30 mL). The extracts were washed with brine and dried over Na₂SO₄. Solvents were evaporated under reduced pressure. The residue was purified by preparative thin layer chromatography eluting with DCM:MeOH (20:1) to give the title compound (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-ol (5). MS-ESI (m/z): 567 [M+1]⁺.

### Example 6

### Ethyl (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)acetate (6)

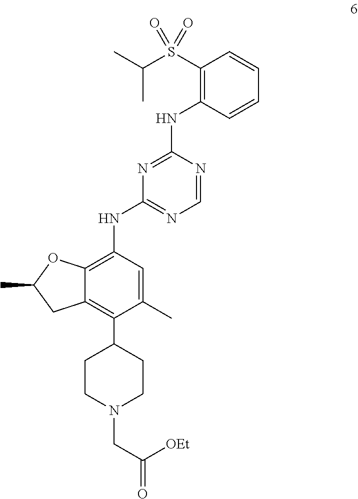 [SMILES: CCOC(=O)CN1CCC(c2c(C)cc(Nc3ncnc(Nc4ccccc4S(=O)(=O)C(C)C)n3)c3c2C[C@@H](C)O3)CC1]

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (30 mg, 0.057 mmol) in anhydrous THF (2 mL) was added ethyl 2-oxoacetate (117 mg, 1.14 mmol) and NaBH(OAc)₃ (60 mg, 0.28 mmol) at 0° C. The mixture was stirred further for 1.5 h. The solvent was removed and the mixture was extracted with ethyl acetate (2×30 mL). The extracts were washed with saturated NaHCO₃ (10 mL), water, brine and dried over Na₂SO₄. Solvents were evaporated under reduced pressure. The residue was purified by preparative thin layer chromatography eluting with DCM:MeOH (30:1) to give the title compound Ethyl (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)acetate (6). MS-ESI (m/z): 609 [M+1]⁺.

### Example 7

### (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-N,N-dimethylacetamide (7)

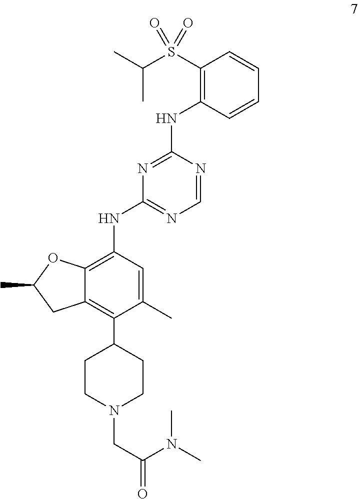 [SMILES: Cc1cc(Nc2ncnc(Nc3ccccc3S(=O)(=O)C(C)C)n2)c2c(c1C1CCN(CC(=O)N(C)C)CC1)C[C@@H](C)O2]

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (30 mg, 0.057 mmol) in DMF (2 mL) at room temperature was added 2-chloro-N,N-dimethylacetamide (13 mg, 0.12 mmol) and K₂CO₃ (24 mg, 0.17 mmol). The mixture was stirred at room temperature for 4 h. The mixture was diluted with water (10 mL) and extracted with ethyl acetate (2×30 mL). The extracts were dried over Na₂SO₄. Solvents were evaporated under reduced pressure. The residue was purified by preparative thin layer chromatography eluting with DCM:MeOH (20:1) to give the title compound (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-N,N-dimethylacetamide (7). MS-ESI (m/z): 608 [M+1]⁺.

### Example 8

### (R)—N²-(2,5-dimethyl-4-(1-(2-(methylsulfonyl)ethyl)piperidin-4-yl)-2,3-dihydrobenzofuran-7-yl)-N⁴-(2-(isopropylsulfonyl)phenyl)-1,3,5-triazine-2,4-diamine (8)

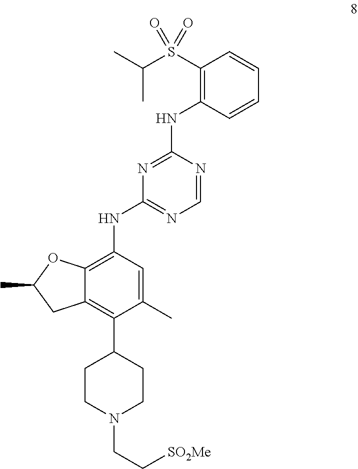 [SMILES: Cc1cc(Nc2ncnc(Nc3ccccc3S(=O)(=O)C(C)C)n2)c2c(c1C1CCN(CCS(C)(=O)=O)CC1)C[C@@H](C)O2]

### (R) N² (4 (1 (2 chloroethyl)piperidin-4-yl)-2,5-dimethyl-2,3-dihydrobenzofuran-7-yl)-N⁴-(2-(isopropylsulfonyl)phenyl)-1,3,5-triazine-2,4-diamine (8a)

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (52 mg, 0.1 mmol) in anhydrous THF (2 mL) was added 2-chloroacetaldehyde (25 mg, 0.3 mmol) and NaBH(OAc)₃ (84 mg, 0.4 mmol) at 0° C. The mixture was stirred further for 1 h. The solvent was removed and the mixture was extracted with ethyl acetate (2×30 mL). The extracts were washed with saturated NaHCO₃ (10 mL), water, brine and dried over Na₂SO₄. Solvents were evaporated under reduced pressure to give the crude product of the title compound (R)—N²-(4-(1-(2-chloroethyl)piperidin-4-yl)-2,5-dimethyl-2,3-dihydrobenzofuran-7-yl)-N⁴-(2-(isopropylsulfonyl)phenyl)-1,3,5-triazine-2,4-diamine (8a). MS-ESI (m/z): 585 [M+1]⁺.

### (R)—N²-(2,5-dimethyl-4-(1-(2-(methylsulfonyl)ethyl)piperidin-4-yl)-2,3-dihydrobenzofuran-7-yl)-N⁴-(2-(isopropylsulfonyl)phenyl)-1,3,5-triazine-2,4-diamine (8)

To a solution of crude (R)—N²-(4-(1-(2-chloroethyl)piperidin-4-yl)-2,5-dimethyl-2,3-dihydrobenzofuran-7-yl)-N⁴-(2-(isopropylsulfonyl)phenyl)-1,3,5-triazine-2,4-diamine (8a) (81 mg) in anhydrous DMF (2 mL) was added sodium methanesulfinate (43 mg, 0.42 mmol) and sodium iodide (15 mg, 0.09 mmol). The reaction mixture was heated to 50° C. for 5 h. The mixture was extracted with ethyl acetate (2×30 mL). The extracts were washed with brine and dried over Na₂SO₄. Solvents were evaporated under reduced pressure. The residue was purified by preparative thin layer chromatography eluting with DCM:MeOH (15:1) to give the title compound (R)—N²-(2,5-dimethyl-4-(1-(2-(methylsulfonyl)ethyl)-piperidin-4-yl)-2,3-dihydrobenzofuran-7-yl)-N⁴-(2-(isopropylsulfonyl)phenyl)-1,3,5-triazine-2,4-diamine (8). MS-ESI (m/z): 629 [M+1]⁺.

### Example 9

### (R)-2-hydroxy-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (9)

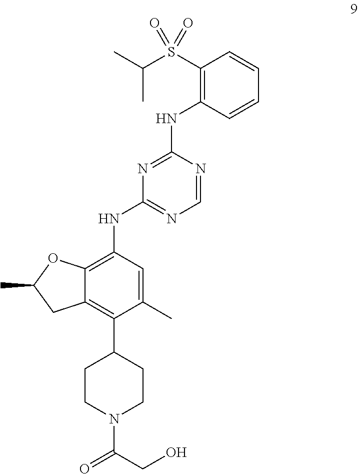 [SMILES: Cc1cc(Nc2ncnc(Nc3ccccc3S(=O)(=O)C(C)C)n2)c2c(c1C1CCN(C(=O)CO)CC1)C[C@@H](C)O2]

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (20 mg, 0.038 mmol) in anhydrous DMF (1.5 mL) was 2-hydroxyacetic acid (6 mg, 0.08 mmol), EDCI (20 mg, 0.1 mmol) and HoBt (14 mg, 0.1 mmol). The mixture was stirred at room temperature for 4 h. The mixture was extracted with ethyl acetate (2×30 mL). The extracts were washed with 1 N NaOH, brine and dried over Na₂SO₄. Solvents were evaporated under reduced pressure. The residue was purified by preparative thin layer chromatography plate eluting with DCM:MeOH (30:1) to give the title compound (R)-2-hydroxy-1-(4-(7-((4-((2-(isopropylsulfonyl)phenyl)amino)-1,3,5-triazin-2-yl)amino)-2,5-dimethyl-2,3-dihydro benzofuran-4-yl)piperidin-1-yl)ethan-1-one (9). MS-ESI (m/z): 581 [M+1]⁺.

### Example 10

### (R)-2-amino-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (10)

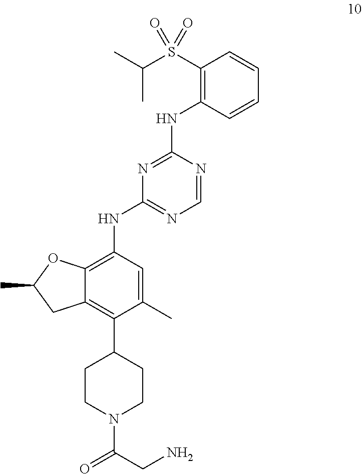 [SMILES: Cc1cc(Nc2ncnc(Nc3ccccc3S(=O)(=O)C(C)C)n2)c2c(c1C1CCN(C(=O)CN)CC1)C[C@@H](C)O2]

### tert-butyl (R)-(2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-2-oxoethyl)carbamate (10a)

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (25 mg, 0.048 mmol) in anhydrous DMF (2 mL) was added (tert-butoxycarbonyl)glycine (13 mg, 0.08 mmol), EDCI (19 mg, 0.1 mmol) and HoBt (13 mg, 0.1 mmol). The mixture was stirred at room temperature for 4 h. The mixture was extracted with ethyl acetate (2×30 mL). The extracts were washed with 1 N NaOH, brine and dried over Na₂SO₄. Solvents were evaporated under reduced pressure to give the crude product of the title compound tert-butyl (R)-(2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-2-oxoethyl)carbamate (10a). MS-ESI (m/z): 680 [M+1]⁺.

### (R)-2-amino-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (10)

To a solution of crude tert-butyl (R)-(2-(4-(7-((4-((2-(isopropylsulfonyl)-phenyl)amino)-1,3,5-triazin-2-yl)amino)-2,5-dimethyl-2,3-dihydrobenzofuran-4-yl)piperidin-1-yl)-2-oxoethyl)carbamate (10a) (61 mg) in anhydrous DCM (3 mL) was added TFA (1 mL). The mixture was stirred at room temperature for 1 h. The mixture was extracted with DCM (2×30 mL), washed with saturated NaHCO₃ (30 mL), water, brine, dried over Na₂SO₄ and concentrated. The residue was purified by preparative thin layer chromatography eluting with DCM:MeOH (10:1) to give the title compound (R)-2-amino-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one (10). MS-ESI (m/z): 580 [M+1]⁺.

### Example 11

### (S)-2-amino-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)propan-1-one (11)

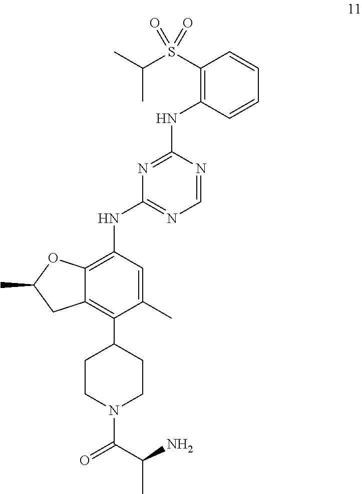 [SMILES: Cc1cc(Nc2ncnc(Nc3ccccc3S(=O)(=O)C(C)C)n2)c2c(c1C1CCN(C(=O)[C@H](C)N)CC1)C[C@@H](C)O2]

### tert-butyl ((S)-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)-1-oxopropan-2-yl)carbamate (11a)

To a solution of (R)—N²-(2,5-dimethyl-4-(piperidin-4-yl)-2,3-dihydrobenzofuran-7-yl)-N⁴-(2-(isopropylsulfonyl)-phenyl)-1,3,5-triazine-2,4-diamine (1) (20 mg, 0.038 mmol) in anhydrous DMF (2 mL) was added (tert-butoxycarbonyl)-L-alanine (8 mg, 0.04 mmol), EDCI (15 mg, 0.08 mmol) and HoBt (10 mg, 0.07 mmol). The mixture was stirred at room temperature for 5 h. The mixture was extracted with ethyl acetate (2×30 mL). The extracts were washed with 1 N NaOH, brine and dried over Na₂SO₄. Solvents were evaporated under reduced pressure to give the crude product of the title compound tert-butyl ((S)-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)-1-oxopropan-2-yl)carbamate (11a). MS-ESI (m/z): 694 [M+1]⁺.

### (S)-2-amino-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)propan-1-one (11)

To a solution of crude tert-butyl ((S)-1-(4-((R)-7-((4-((2-(isopropylsulfonyl)-phenyl)amino)-1,3,5-triazin-2-yl)amino)-2,5-dimethyl-2,3-dihydrobenzofuran-4-yl)piperidin-1-yl)-1-oxopropan-2-yl)carbamate (11a) (60 mg) in anhydrous DCM (1 mL) was added TFA (0.5 mL). The mixture was stirred at room temperature for 1.5 h. The mixture was extracted with DCM (2×30 mL), washed with saturated NaHCO₃ (20 mL), water, brine, dried over Na₂SO₄ and concentrated. The residue was purified by preparative thin layer chromatography eluting with DCM:MeOH (10:1) to give the title compound (S)-2-amino-1-(4-((R)-7-((4-((2-(isopropylsulfonyl)phenyl)amino)-1,3,5-triazin-2-yl)amino)-2,5-dimethyl-2,3-dihydro benzofuran-4-yl)piperidin-1-yl)propan-1-one (11). MS-ESI (m/z): 594 [M+1]⁺.

### Cell Proliferation Assays

NCI-H3122 or SUP-M2 Cells were plated into 96-well plates (3000-8000/well) in complete medium. After incubation overnight, cells were exposed to various concentrations of compound for further 72 h. Cell proliferation was evaluated by SRB assay (for NCI-H3122 cell) or Cell Counting Kit-8 assay (CCK-8, DOJINDO Molecular Technologies, Inc.) (for SUP-M2 cell). ICso values were calculated by concentration-response curve fitting using a SoftMax pro-based four-parameter method. Each IC₅₀ value was expressed as mean±SD from three separate experiments.

Select compounds prepared as described above were assayed according to the biological procedures described herein. The results are given in the Table 1.

TABLE 1

IC₅₀ (nM)

| Example | SUP-M2 | H3122 |
|---|---|---|
| 1 | 99.3 | 52.6 |
| 2 | 54.4 | 20.8 |
| 3 | 50.4 | 14.4 |
| 4 | 111-333 | / |
| 5 | 70.0 | 57.7 |
| 6 | 111-333 | / |
| 7 | 111-333 | / |
| 8 | 111-333 | / |
| 9 | 33.7 | 15.9 |
| 10 | 79.6 | 133.6 |
| 11 | 67.1 | 25.7 |

“/” denotes that it was not measured.

All references cited herein are incorporated by reference in their entirety.

What is claimed is:

1. A compound of formula (I): 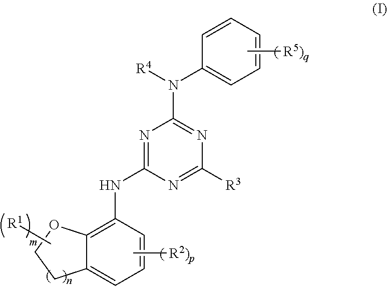
or a pharmaceutically acceptable salt thereof, wherein:
each R¹ is independently selected from hydrogen, halogen, hydroxyl, C₁₋₁₀ alkyl (such as C₁₋₆ alkyl), C₃₋₁₀ cycloalkyl (such as C₃₋₆ cycloalkyl), C₃₋₁₀ cycloalkyl-C₁₋₄ alkyl, heterocyclyl, heterocyclyl-C₁₋₄ alkyl, aryl, aryl-C₁₋₄ alkyl, heteroaryl, and heteroaryl-C₁₋₄ alkyl, wherein alkyl, cycloalkyl, and heterocyclyl are each unsubstituted or substituted with at least one substituent, such as one, two, three, or four substituents, independently selected from R^{6a}, and wherein aryl and heteroaryl are each unsubstituted or substituted with at least one substituent, such as one, two, three, or four substituents, independently selected from R^{6b};
each R² is independently selected from hydrogen, halogen, hydroxyl, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, heterocyclylcarbonyl, aryl, heteroaryl, aryl-C₁₋₄ alkyl, and heteroaryl-C₁₋₄ alkyl, wherein alkyl, alkenyl, alkynyl, cycloalkyl, and heterocyclyl are each unsubstituted or substituted with at least one substituent, such as one, two, three, or four substituents, independently selected from R^{6a}, and each aryl and heteroaryl is unsubstituted or substituted with at least one substituent, such as one, two, three, or four substituents, independently selected from R^{6b};
each R³ is independently selected from hydrogen, halogen, —CN, —NR⁷R⁸, and C₁₋₁₀ alkyl; wherein alkyl is unsubstituted or substituted with at least one substituent, such as one, two, three, or four substituents, independently selected from R^{6a};
each R⁴ is independently selected from hydrogen, halogen, —CN, C₁₋₁₀ alkyl, C₂₋₁₀ alkenyl, C₂₋₁₀ alkynyl, and C₃₋₁₀ cycloalkyl; wherein alkyl, alkenyl, alkynyl, and cycloalkyl are each unsubstituted or substituted with at least one substituent, such as one, two, three, or four substituents, independently selected from R^{6a};
or R³ and R⁴ together with the carbon atoms to which they are attached form a 5-6 membered ring containing 0, 1, 2 or 3 heteroatoms independently selected from oxygen, sulfur and nitrogen, and optionally substituted with 1-2 R^{6b} groups;
each R⁵ is independently selected from hydrogen, C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃; wherein alkyl, alkenyl, alkynyl, and cycloalkyl are each unsubstituted or substituted with at least one substituent, such as one, two, three, or four substituents, independently selected from R^{6a};
each R^{6a} is independently selected from C₁₋₁₀ alkyl, C₂₋₁₀ alkenyl, C₂₋₁₀ alkynyl, C₃₋₁₀ cycloalkyl, —OR⁸, —NR⁷S(O)ᵣR⁸, —NO₂, halogen, —S(O)ᵣR⁷, —SR⁸, —S(O)₂OR⁷, —OS(O)₂R⁸, —S(O)ᵣNR⁷R⁸, —NR⁷R⁸, —(CR⁹R¹⁰)ₜOR⁸, —(CR⁹R¹⁰)ₜNR⁷R⁸, —(CR⁹R¹⁰)ₜSR⁸, —(CR⁹R¹⁰)ₜS(O)ᵣR⁸, —(CR⁹R¹⁰)ₜCO₂R⁸, —(CR⁹R¹⁰)ₜCONR⁷R⁸, —(CR⁹R¹⁰)ₜNR⁷CO₂R⁸, —(CR⁹R¹⁰)ₜOCONR⁷R⁸, —(CR⁹R¹⁰)ₜNR⁷CONR⁷R⁸, —(CR⁹R¹⁰)ₜNR⁷SO₂NR⁷R⁸, —O(CR⁹R¹⁰)ₜNR⁷R⁸, —C(O)R⁷, —C(O)(CR⁹R¹⁰)ₜOR⁸, —C(O)(CR⁹R¹⁰)ₜNR⁷R⁸, —C(O)(CR⁹R¹⁰)ₜSR⁸, —C(O)(CR⁹R¹⁰)ₜS(O)ᵣR⁸, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CR⁷(N—OR⁸), —CHF₂, —CF₃, —OCHF₂, and —OCF₃;
each R^{6b} is independently selected from R^{6a}, aryl, aryl-C₁₋₄ alkyl, heteroaryl, and heteroaryl-C₁₋₄ alkyl;
each R⁷ and each R⁸ are independently selected from hydrogen, C₁₋₁₀ alkyl, C₂₋₁₀ alkenyl, C₂₋₁₀ alkynyl, cycloalkyl, cycloalkyl-C₁₋₄ alkyl; heterocyclyl, heterocyclyl-C₁₋₄ alkyl, aryl, heteroaryl, aryl-C₁₋₄ alkyl, and heteroaryl-C₁₋₄ alkyl; wherein alkyl, alkenyl, alkynyl, cycloalkyl, and heterocyclyl are each unsubstituted or substituted with at least one substituent, such as one, two, three, or four substituents, independently selected from R^{6a}, and aryl and heteroaryl are each unsubstituted or substituted with at least one substituent, such as one, two, three, or four substituents, independently selected from R^{6b}; or
R⁷ and R⁸ together with the atom(s) to which they are attached form a heterocyclic ring of 4 to 7 members containing 0, 1, or 2 additional heteroatoms independently selected from oxygen, sulfur and NR¹¹, each R⁷ and R⁸ may be unsubstituted or substituted on a carbon or nitrogen atom with at least one substituent, such as one, two, or three substituents, selected from R¹²;
each R⁹ and each R¹⁰ are independently selected from: hydrogen, C₁₋₁₀ alkyl, C₂₋₁₀ alkenyl, C₂₋₁₀ alkynyl, cycloalkyl, cycloalkyl-C₁₋₄ alkyl, heterocyclyl, heterocyclyl-C₁₋₄ alkyl, aryl, heteroaryl, aryl-C₁₋₄ alkyl, and heteroaryl-C₁₋₄ alkyl; or
R⁹ and R¹⁰ together with the carbon atom(s) to which they are attached form a ring of 3 to 7 members containing 0, 1, or 2 heteroatoms independently selected from oxygen, sulfur and nitrogen, and optionally substituted with 1-2 R^{6a} groups;
each R¹¹ is independently selected from hydrogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —S(O)ᵣR⁷, —C(O)R⁷, —CO₂R⁷, —CO₂(CR⁹R¹⁰)ₜCONR⁷R⁸, and —C(O)NR⁷R⁸;
each R¹² is independently selected from halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, —OR⁷, —NR⁷S(O)ᵣR⁸, —S(O)ᵣR⁷, —SR⁷, —S(O)₂OR⁷, —OS(O)₂R⁷, —S(O)ᵣNR⁷R⁸, —NR⁷R⁸, —O(CR⁹R¹⁰)ₜNR⁷R⁸, —C(O)R⁷, —CO₂R⁸, —CO₂(CR⁹R¹⁰)ₜCONR⁷R⁸, —OC(O)R⁷, —CN, —C(O)NR⁷R⁸, —NR⁷C(O)R⁸, —OC(O)NR⁷R⁸, —NR⁷C(O)OR⁸, —NR⁷C(O)NR⁷R⁸, —CHF₂, —CF₃, —OCHF₂, and —OCF₃;
each m is independently selected from 0, 1 and 2;
each n is independently selected from 1, 2, and 3;
each p is independently selected from 0, 1, 2, and 3;
each q is independently selected from 0, 1, 2, and 3;
each r is independently selected from 1 and 2;
each t is independently selected from 1, 2, and 3.

2. A compound of claim 1 or a pharmaceutically acceptable salt thereof, wherein R² is selected from C₁₋₁₀ alkyl and heterocyclyl, wherein alkyl and heterocyclyl are each unsubstituted or substituted with at least one substituent, such as one, two, three, or four substituents, independently selected from R^{6a}, and R^{6a}.

3. A compound of claim 2 or a pharmaceutically acceptable salt thereof, wherein R² is selected from methyl and piperidinyl, wherein piperidinyl is unsubstituted or substituted with at least one substituent, such as one, two, three, or four substituents, independently selected from R^{6a}, and R^{6a}.

4. A compound of claim 3 or a pharmaceutically acceptable salt thereof, wherein the substructure of Formula (II): 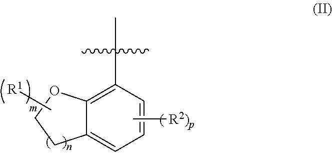
in Formula (I) is

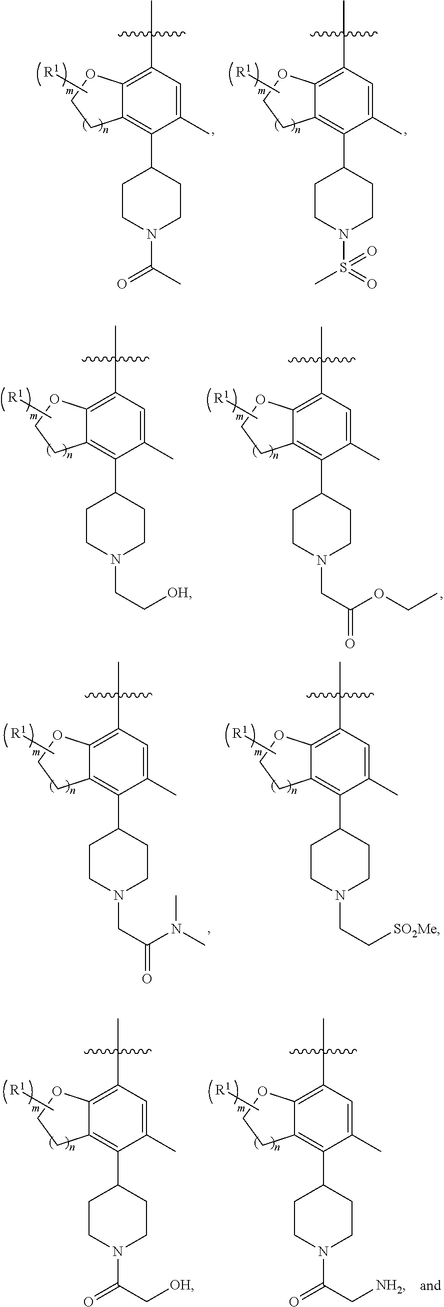

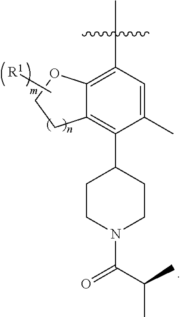

5. A compound of claim 1 or a pharmaceutically acceptable salt thereof, wherein R¹ is C₁₋₁₀ alkyl.

6. A compound of claim 5 or a pharmaceutically acceptable salt thereof, wherein R¹ is methyl and m=1.

7. A compound of claim 1 or a pharmaceutically acceptable salt thereof, wherein R⁵ is —S(O)ᵣR⁷.

8. A compound of claim 7 or a pharmaceutically acceptable salt thereof, wherein substructure of Formula (III): 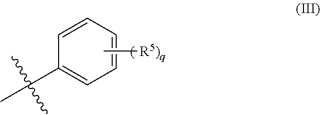
in Formula (I) is

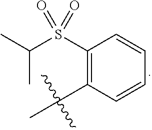 [SMILES: *c1ccccc1S(=O)(=O)C(C)C]

9. A compound of claim 1 or a pharmaceutically acceptable salt thereof, wherein R³ is hydrogen.

10. A compound of claim 1 or a pharmaceutically acceptable salt thereof, wherein R⁴ is hydrogen.

11. A compound of claim 1 or a pharmaceutically acceptable salt thereof, wherein n is 1.

12. A compound selected from
(R)—N²-(2,5-dimethyl-4-(piperidin-4-yl)-2,3-dihydrobenzofuran-7-yl)-N⁴-(2-(isopropylsulfonyl)phenyl)-1,3,5-triazine-2,4-diamine,
(R)—N²-(2,5-dimethyl-4-(1-methylpiperidin-4-yl)-2,3-dihydrobenzofuran-7-yl)-N⁴-(2-(isopropylsulfonyl)phenyl)-1,3,5-triazine-2,4-diamine,
(R)-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one
(R)—N²-(2,5-dimethyl-4-(1-(methylsulfonyl)piperidin-4-yl)-2,3-dihydrobenzofuran-7-yl)-N⁴-(2-(isopropylsulfonyl)phenyl)-1,3,5-triazine-2,4-diamine,
(R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-ol,
ethyl (R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)acetate,
(R)-2-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)-N,N-dimethylacetamide,
(R)—N²-(2,5-dimethyl-4-(1-(2-(methylsulfonyl)ethyl)piperidin-4-yl)-2,3-dihydrobenzofuran-7-yl)-N⁴-(2-(isopropylsulfonyl)phenyl)-1,3,5-triazine-2,4-diamine,
(R)-2-hydroxy-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one,
(R)-2-amino-1-(4-(7-((4-((2-(isopropylsulfonyl)phenyl)amino)-1,3,5-triazin-2-yl)amino)-2,5-dimethyl-2,3-dihydrobenzofuran-4-yl)piperidin-1-yl)ethan-1-one,
(S)-2-amino-1-(4-((R)-7-((4-((2-(isopropylsulfonyl)phenyl)amino)-1,3,5-triazin-2-yl)amino)-2,5-dimethyl-2,3-dihydrobenzofuran-4-yl)piperidin-1-yl)propan-1-one,
or a pharmaceutically acceptable salt thereof.

13. A method of treating, ameliorating or preventing a condition, which responds to inhibition of anaplastic lymphoma kinase, comprising administering to a subject in need of such treatment an effective amount of a compound of claim 1, or a pharmaceutically acceptable salt thereof, or at least one pharmaceutical composition thereof, and optionally in combination with a second therapeutic agent.
